(19) United States
(12) Patent Application Publication
(10) Pub. No.: US 2004/0039344 A1
(43) Pub. Date: Feb. 26, 2004

(54) System, method and apparatus for administering medical liquids

(76) Inventors: Brian Eugene Baldwin, Centennial, CO (US); Joseph V. Ranalletta, Englewood, CO (US)

Correspondence Address: MARSH FISCHMANN & BREYFOGLE LLP, Suite 411, 3151 South Vaughn Way, Aurora, CO, 80014, US

(21) Appl. No.: 10/226,192

(22) Filed: Aug. 22, 2002

(51) Int. Cl. A61M 5/00

(52) U.S. Cl. 604/209

(57) ABSTRACT

A system, method and apparatus are provided for administering medical liquids to a patient. The invention is particularly apt for use in the intravenous administration of multiple doses of a flush solution over an extended period of therapy. In certain aspects of the invention methods and apparatus are directed to the provision and utilization of a packaged syringe filled with a medical liquid and having a tubing length interconnected thereto, wherein the volume of pre-filled medical liquid is sufficient to allow for multiple administrations to a given patient. By way of primary example, where the syringe is pre-filled with a flush solution, a volume of at least about 40 ml. is preferred. In other aspects, methods and apparatus are directed to the administration of medical liquids from a pre-filled syringe utilizing an apparatus that is particularly adapted for administering a medical liquid in successive increments over a plurality of separated time intervals.

### RELATED APPLICATIONS

The present invention relates to a system for dispensing flowable materials, and more particularly to methods and apparatus for use in connection with the intravenous administration of medical liquids to a patient. The inventive aspects are particularly apt for use in applications entailing vascular catheter flushing on multiple occasions over an extended period of patient care.

### BACKGROUND OF THE INVENTION

Numerous techniques are employed for the administration of “medical liquids” (e.g. liquid medication and flush solutions) to a patient. In particular, where repeated medication infusions are required, medical liquids are often administered via the use of a vascular access catheter that is fluidly interconnected or interconnectable to one or more medical liquid sources via an associated tubing line set. Typically, the catheter is inserted into the vein of a patient and left there for multiple intravenous (IV) infusions during an extended course of medication therapy. By way of example, the time period between IV drug infusions may be between about 4 to 24 hours, wherein the IV liquid medication source is typically replaced after each dose infusion. In the course of extended medication therapy a given tubing line set may be repeatedly employed, and a number of tubing line sets may be successively employed. For example, it is typical to replace a given tubing line set every two or three days.

During extended therapy applications, a desirable practice is to disconnect the vascular catheter from a medical liquid source and tubing line set between infusions. In this regard, most patients receiving IV medication therapy are ambulatory to some degree and benefit from not being continuously connected.

In conjunction with the repeated connection/disconnection of a vascular catheter and liquid medication source and tubing line set, it is usual practice to purge the vascular catheter with a flush solution (e.g. a saline solution) prior to and at the completion of a given liquid medication infusion. Pre-infusion flushing verifies that the vascular catheter is primed and clear of obstructions. Post infusion flushing not only flushes through any remaining liquid medication to achieve the desired therapeutic effect, but also reduces any chance that the vascular catheter may become blocked in-between infusions, e.g. by a blood clot that may otherwise form in the vascular catheter. In relation to infusion procedures, it is also common practice to verify the proper functioning of a vascular catheter via aspiration. This is typically done prior to pre-infusion flushing and after liquid medication infusion. The procedure entails using the flush solution syringe or liquid medication syringe to drain a small amount of a patient's blood through the vascular catheter, thereby permitting visual verification of proper vascular catheter functionability, then advancing the blood back through the vascular catheter using the syringe. By way of example, such procedure assures that the vascular catheter is not blocked by a blood clot and is otherwise properly inserted into a patient's vascular system.

A number of approaches are currently utilized for the noted flushing procedures. Such techniques generally entail the usage of flush solutions packaged in large volume, multi-dose reservoirs (e.g. about 250 ml. or more) or pre-filled unit dose syringes (e.g. having volumes of 2, 3, 5 or 10 ml.).

Where a unit dose syringe is utilized, medical personnel must generally remove the syringe from packaging, remove a cap from the syringe, remove any air in the syringe, swab a vascular catheter access port with an antibacterial material, interconnect the syringe to a vascular catheter access port, optionally aspirate the vascular catheter, advance the syringe plunger to infuse the flush solution (e.g. at a rate of about 5 to 10 ml. over about 15 to 30 seconds), remove the syringe from the vascular catheter access port and discard the used syringe with its wrapper. As may be appreciated, such steps may need to be repeated numerous times over the course of extended medication therapy, e.g. after each infusion and vascular catheter access port reconnection, thereby entailing significant medical personnel time and resulting in substantial medical waste. Further, while unit dose syringes provide good sensitivity for aspiration purposes they are not particularly pressure sensitive for flushing purposes.

Where multi-dose flush solution reservoirs are employed, medical personnel typically utilize an empty unit dose syringe to draw the flush solution from the reservoir, then follow the same basic procedure noted above in an administering the flush solution. Again, such procedure may be followed a number of times during a medication therapy. Further, contamination concerns may arise when a unit dose syringe is filled from a multi-dose reservoir at the point of use. To address such concern, unit dose syringes are often filled from a multi-dose reservoir within a pharmacy department of a medical care facility utilizing a hepa-filter air hood. However, significant syringe handling is required. Moreover, labeling becomes a further need when a delay is expected between the filling of a unit dose syringe and the usage of the filled syringe.

### SUMMARY OF THE INVENTION

In view of the foregoing, primary objectives of the present invention are to reduce the number of steps, complexity and associated time required by medical personnel for the infusion of medical liquids, and in particular flush solutions, in conjunction with IV procedures. More particularly, a specific objective is to reduce the number of connections/disconnections made for flushing purposes in relation to each liquid medication infusion.

An additional objective of the present invention is to provide one or more of the noted medical liquid administration benefits in a manner that also enhances the maintenance of sterility, thereby reducing any risk of infection.

Yet another objective of the present invention is to reduce medical waste associated with the IV administration of flush solutions.

The above objectives and additional advantages are met by various aspects of the present invention. Such aspects comprise methodology and apparatus directed to the repeated administration of a medical liquid from a single source to a patient.

In one aspect, an inventive method is provided that includes the steps of filling a syringe with a medical liquid and providing a tubing length for interconnection to the syringe. The method further includes the step of packaging the pre-filled syringe and tubing length, e.g. for subsequent transport, storage and use. To facilitate use after unpackaging, the tubing length may be interconnected to the pre-filled syringe prior to packaging. Preferably, the tubing length is also provided with at least one outlet connector at its distal end for selective interconnection with a vascular catheter access port to a patient.

In one embodiment a male luer connector is provided at the distal end of the tubing length. In another embodiment a Y-site member is provided at the distal end, wherein the Y-site member includes a male luer connector outlet and female luer connector inlet (e.g. for selective fluid interconnection to a vascular catheter access port and a liquid medication source, respectively).

The medical liquid may comprise a volume of medical liquid, e.g. between about 10 ml. and 200 ml. More particularly, the present inventors have recognized that to satisfy vascular catheter flushing needs of a given patient in connection with successive infusions of liquid medication over an extended time period the syringe may be pre-filled with a flush solution volume of at least about 40 ml., and most preferably about 60 ml. Such volume allows for repeated vascular catheter flushing, e.g. both prior and subsequent to liquid medication infusion to insure vascular catheter functionality. In the later regard, vascular catheter flushing prior to drug infusion is particularly desirable in conjunction with procedures where a vascular catheter and liquid medication source are disconnected/reconnected between successive infusions.

In relation to the noted method, the inventors have further recognized the desirability of maintaining a relatively low flow rate at a vascular catheter during catheter flushing. As such, the noted tubing length is preferably of a “microbore” type (e.g. having an inside diameter of between about 0.75 mm and 1.50 mm).

The noted method may further include the step of sterilizing the pre-filled syringe and tubing length. In this regard, the sterilizing step may entail exposing the pre-filled syringe and tubing length to gamma radiation. Moreover, the syringe and tubing length may be exposed simultaneously to gamma radiation (e.g. after interconnection). Where gamma radiation is utilized, sterilization may occur either prior to or after the packaging step.

As will be understood, the inventive apparatus corresponding with the noted method comprises a syringe filled with a medical liquid (e.g. a flush solution) and a tubing length that may be interconnected to the pre-filled syringe prior to packaging. At least one connector may be interconnected to a distal end of the tubing length. Such connector may be adapted and otherwise sized for ready interconnection to and disconnection from a vascular catheter access port. In one embodiment, the connector may be a male luer connector. Correspondingly, a protective screw-on/off cap may be provided on the connector. In another embodiment, a distal Y-site member is provided having both a male luer outlet connector and a female luer inlet connector, each of which may be provided with screw-on/off caps.

The apparatus may further include an enclosure within which the pre-filled syringe and tubing length are sealably disposed, e.g. prior to transport, storage and use. Such enclosure may comprise a material that facilitates ready heat sealing. Further, the enclosure material may be of a type that accommodates the utilization of gamma radiation for sterilization of the pre-filled syringe and tubing length after placement within the packaging.

As noted above, the medical liquid may comprise a volume of flush solution that is preferably at least about 40 ml., and most preferably about 60 ml. By way of example, the flush solution may be a saline solution or heparin-containing solution.

In another aspect of the present invention, an inventive method is provided for use in the administration of medical liquids through a vascular catheter. The method comprises a first step of removing a syringe and tubing length from a sealed package, wherein the syringe is pre-filled with a medical liquid. The tubing length may be pre-connected to the pre-filled syringe, thereby facilitating use upon unpackaging. Alternatively, the tubing length may be interconnected to the pre-filled syringe after unpackaging. Preferably, an outlet connector is interconnected to a distal end of the tubing length for ready interconnection with a vascular catheter access port.

The method further includes the steps of fluidly interconnecting the tubing length to an access port to a vascular catheter inserted in a patient (e.g. via use of the noted connector), and administering medical liquid from the syringe to the patient through the tubing length and vascular catheter. For extended therapy applications the administering step may include the dispensing of successive increments of the medical liquid over a plurality of separated time intervals. In this regard, the administration of the medical liquid may entail the progressive advancement of a plunger into the barrel of the syringe over the course of the separated time intervals.

In primary applications, the medical liquid comprises a catheter flush solution, wherein the administering step may provide for the administration of between about 2 ml. and 10 ml. of flush solution, per occasion. In this regard, the method may also include the further step of infusing a liquid medication to the patient via the vascular catheter between each of the noted time intervals during which the flush solution is administered. In particular, the inventive method may provide for the flushing of a vascular catheter from the pre-filled syringe after and/or prior to each liquid medication infusion that is administered through the vascular catheter. Of note, when a Y-site member is provided at the distal end of the tubing length the flush solution administration and liquid medication infusion steps may each be repeated a plurality of times while advantageously maintaining the interconnections between the pre-filled syringe and the Y-site member and between the liquid medication source and the Y-site member.

In another aspect of the present invention, an administration apparatus is provided for administering a medical liquid from a syringe, such as the pre-filled syringe noted above, when interconnected to a vascular catheter. The apparatus includes a first holder member for restrainably engaging one of a plunger and cylinder of a syringe, and a second holder member for restrainably engaging the other of the plunger and cylinder of a syringe. Additionally, the second holder member may be spring-loaded towards the first holder member, and the apparatus may include a drive means for mechanically driving the first holder member towards the second holder member along a travel path. As will be further described, the combination of a mechanical drive means and an opposed spring-loading yields an easy to use apparatus capable of administering successive increments of a medical liquid (e.g. a flush solution) from a syringe over an extended time period.

The spring-loading of the second holder members is preferably established to be at least about 5 psi, and most preferably between about 10 psi and 15 psi. Further, the second holder member may be provided to permit a user to manually move the second holder member away from the first holder member against the noted spring-loading. More particularly, a least one lever arm may be pivotably disposed to effect movement of the second holder member towards the first holder member. As will be further described, such an arrangement provides a means for highly sensitive aspiration of a vascular catheter that is interconnected to a syringe employed with the apparatus.

In one arrangement, the inventive apparatus includes a housing to which the second holder member is pivotably interconnected, and at least one spring member for providing the spring-loading of the second holder member towards the first holder. Preferably, the second holder member is pivotable to travel a distance (e.g. relative to the housing) that is at least as great as the distance traveled by the first holder member (e.g. relative to the housing) in conjunction with the administration of a given increment of medical liquid.

To further facilitate the incremental administration of medical liquid utilizing the inventive apparatus, the apparatus may further include stop means for restricting movement of the first holder member at a plurality of spaced positions along the travel path. By way of primary example, the stop means may include a plurality of stops corresponding with the plurality of stop positions and at least one catch member for restrainably and successively engaging the plurality of stops. Preferably, the plurality of stops may be located along the travel path and the catch member may be interconnected to the first holder member and spring-loaded towards the plurality of stops. In the later regard, the spring-loading of the catch member may be established at between about 2 psi and 5 psi, and most preferably about 3 psi, wherein the catch member may be manually disengaged relative to the stops.

In one arrangement, the catch member may be a pawl that is pivotably interconnected to the first holder member. In turn, the stop means may further include an actuator member for pivoting the pawl away from the plurality of stops against the spring-loading thereof.

In one embodiment, the drive means of the inventive apparatus includes a first drive arm pivotably carried by the first holder member to effect movement of the first holder member between successive ones of said plurality of stop positions (e.g. upon being pivoted by a user). Preferably, the first drive arm is provided so that a restricted pivot range, or stroke length, is defined, wherein a full stroke moves the first holder member a first predetermined number of stop positions (e.g. one or more) to effect the administration of a first predetermined increment of medical liquid.

In the noted embodiment, the drive means may further include a drive shaft rotatably supported by the first holder member and a ratchet means supported by the drive shaft and interconnected to the first drive arm. The ratchet means may function to translate pivotable movement of the first drive arm between first and second positions (e.g. defining a full stroke length therebetween) to rotational movement of the drive shaft. The ratchet means may further function to automatically return the first drive arm from its second position to its first position without rotating the drive shaft.

The drive means may further include a second drive arm that is also pivotally carried by the first holder member to effect movement of the first holder member between successive ones of said plurality of stop positions (e.g. upon being pivoted by a user). Again, the second drive arm is provided so that a restricted pivot range, or stroke length, is defined, wherein a given full stroke moves the second holder member a second predetermined number of stop positions (e.g. one or more) to effect the administration of a second predetermined increment of medical liquid. Of note, the stroke lengths of the first and second drive arms may be advantageously established to be different, wherein the first and second predetermined number of stop positions moved per stroke are different, as well as the resulting first and second predetermined increments of medical liquid administered from a syringe employed with the apparatus. In one arrangement the first and second increments may be established at 2.5 ml. and 5 ml.

The second drive arm also may be connected to the ratchet means, wherein the ratchet means functions to translate pivotable movement of the second drive arm between first and second positions (e.g. defining a full stroke length therebetween) to rotational movement of the drive shaft. Again, the ratchet means may further function to automatically return the second drive arm from its second position to its first position without rotating the drive shaft.

In another embodiment, the drive means may include a drive spring that is activatable to apply a spring force to the first holder member to effect movement thereof across the plurality of stop positions upon a single activation of the drive spring. In one approach, the drive spring may be disposed to be in tension upon activation. In such approach, a negator-type spring may advantageously employed. In another approach, the drive spring may be disposed in compression upon activation. In either case, the spring member may be actuated a single time upon loading of a syringe into the inventive apparatus. Subsequently, the spring member may be successively employed to apply a spring force to effect incremental administration of medical liquid from the syringe by the apparatus.

In this embodiment, the inventive apparatus may be provided so that the stop means is controllable to provide at least two different spacings between the plurality of noted stop positions. Such control allows at least two different increments of medical liquid administration to be achieved. More particularly, first and second sets of stops may be provided, wherein a first spacing between the first set of stops is different from a second spacing between the second set of stops. In turn, a user may selectively employ the first or second set of stops in use of the inventive apparatus.

As may be appreciated, one or more of the above-noted aspects may be implemented in an overall system for providing a medical liquid to a patient. In one embodiment, the system may comprise the steps of packaging a syringe filled with a flush solution at a first location (e.g. a location where the syringe is filled with the flush solution), packaging the filled syringe at a second location (e.g. a patient care site), positioning the filled syringe in an administration apparatus at the second location, and utilizing the administration apparatus to control the administration of the flush solution to a patient through a vascular catheter in a plurality of successive increments over a corresponding plurality of separated time intervals.

As may be appreciated, the system may include the further step of interconnecting a tubing length to the syringe prior to the packaging step. Preferably, an outlet connector is also provided at a distal end of the tubing length. Further, the syringe may be filled with at least about 40 ml. of the flush solution prior to the packaging step. Additionally, the filled syringe and interconnected tubing length may be sterilized (e.g. via exposure to gamma radiation) prior to said unpackaging step (e.g. after said packaging step and prior to transport to a patient care site).

In conjunction with the utilization of an administration apparatus, the system may employ the administration apparatus to progressively drive the syringe plunger into the syringe barrel over the plurality of separated time intervals. For such purposes, the syringe plunger and syringe barrel may be restrainably positioned in first and second holder members, respectively, comprising the administration apparatus. In turn, at least one of the first and second holder members may be employed to effect relative movement between the syringe plunger and syringe barrel, wherein the syringe plunger is incrementally advance(s) within the syringe barrel to administer the flush solution.

As will be appreciated, the system may also include the steps of interconnecting and disconnecting an outlet connector provided at the distal end of the tubing length a vascular catheter access port. For example, a male luer connector allows for easy connection/ disconnection.

In relation to the noted system, a liquid medication may be infused to a patient through a vascular catheter between the plurality of separated time intervals of flushing. In conjunction with liquid medication infusion, the system may further include the steps of: (i) first administering an increment of the flush solution prior to liquid medication infusion, and (ii) second administering an increment of the flush solution after liquid medication infusion. Of note, the steps of infusing a liquid medication and administering a flush solution through the vascular catheter may be repeated a plurality of times while conveniently maintaining the noted positioning of the syringe in the administration apparatus.

Further, in one arrangement, liquid medication infusion may be completed via selective interconnection of a liquid medication source to a Y-site member provided at the distal end of the above-noted tubing length. In turn, the flush solution administration and liquid medication infusion steps may each be repeated a plurality of times while advantageously maintaining the interconnections between the pre-filled syringe and the Y-site member and between the liquid medication source and the Y-site member. The maintenance of such interconnections simplifies the procedure and enhances the maintenance of sterility.

In later regard, the noted system and methods may also provide for the sterile docking of a male inerconnector provided at the distal end of the tubing length attached to the pre-filled syringe. By way of example, a docking apparatus and method may be employed as taught in a U.S. patent application entitled “STERILE DOCKING APPARATUS AND METHOD”, filed on Aug. 22, 2002, the entirety of which is hereby incorporated by reference.

Additional aspects and advantages of the present invention will become apparent to those skilled in the art upon consideration of the further description provided hereinbelow.

### BRIEF DESCRIPTION OF THE DRAWINGS

FIG. 1A illustrates one embodiment of a pre-filled syringe assembly comprising the present invention.

FIG. 1B illustrates the embodiment of FIG. 1A as packaged for transport and storage.

FIG. 2A illustrates another embodiment of a pre-filled syringe assembly comprising the present invention.

FIG. 2B illustrates the embodiment of FIG. 1B as packaged for transport and storage.

FIG. 3. illustrates one embodiment of a method for providing a pre-filled syringe assembly according to the present invention.

FIG. 4 illustrates one embodiment of a method for use in the administration of a medical liquid to a patient.

FIG. 5 illustrates further optional steps for use in connection with the method embodiment of FIG. 4.

FIG. 6 illustrates one embodiment of an apparatus for use in administering medical liquid from the syringe assembly embodiment of FIGS. 1A and 1B.

FIG. 7 is another view of the apparatus and syringe assembly embodiments shown in FIG. 6.

FIG. 8 is a cross-section side view of the apparatus and syringe assembly embodiments shown in FIG. 6.

FIGS. 9A, 9B, 9C and 9D are perspective, top, front and rear views of the apparatus and syringe assembly embodiments shown in FIG. 6.

FIG. 10 illustrates another embodiment of an administration apparatus for use with the syringe assembly embodiments of FIGS. 1A and 1B.

FIG. 11 is another view of the apparatus and syringe assembly embodiments as shown in FIG. 10.

FIG. 12 is a cross-sectional side view of the apparatus and syringe assembly embodiments as shown in FIG. 10.

FIG. 13 is a cross-sectional side view of the dispenser assembly embodiment and syringe assembly embodiment as shown in FIG. 6.

### DETAILED DESCRIPTION

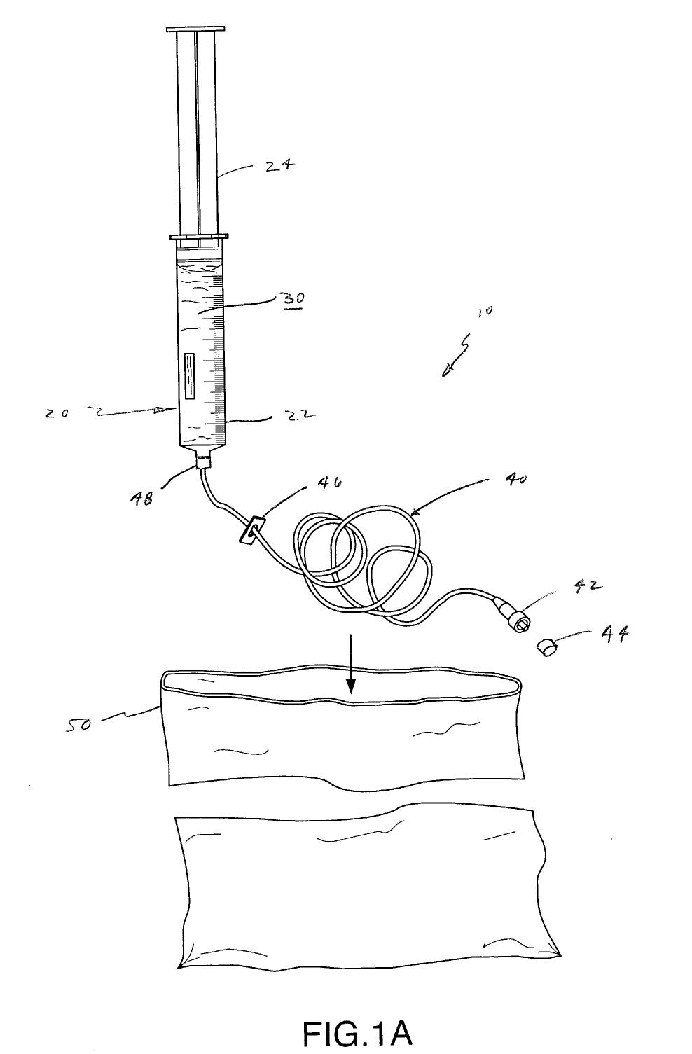

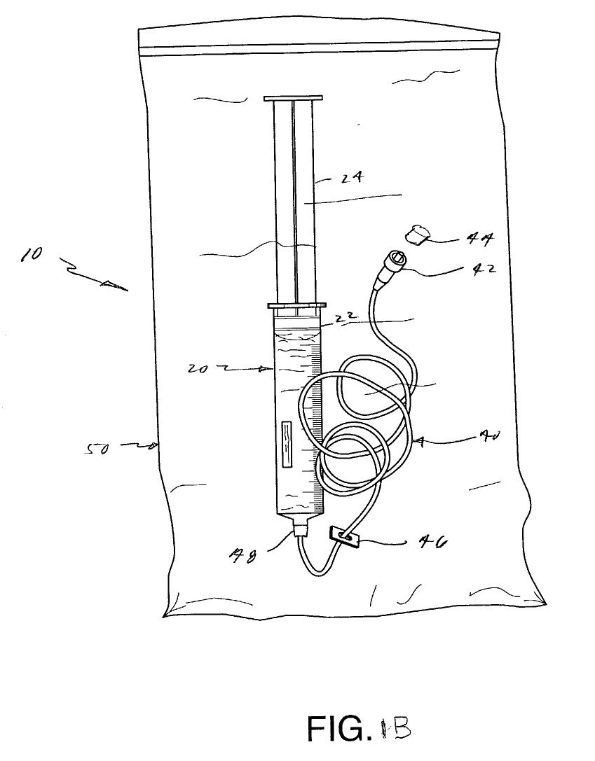

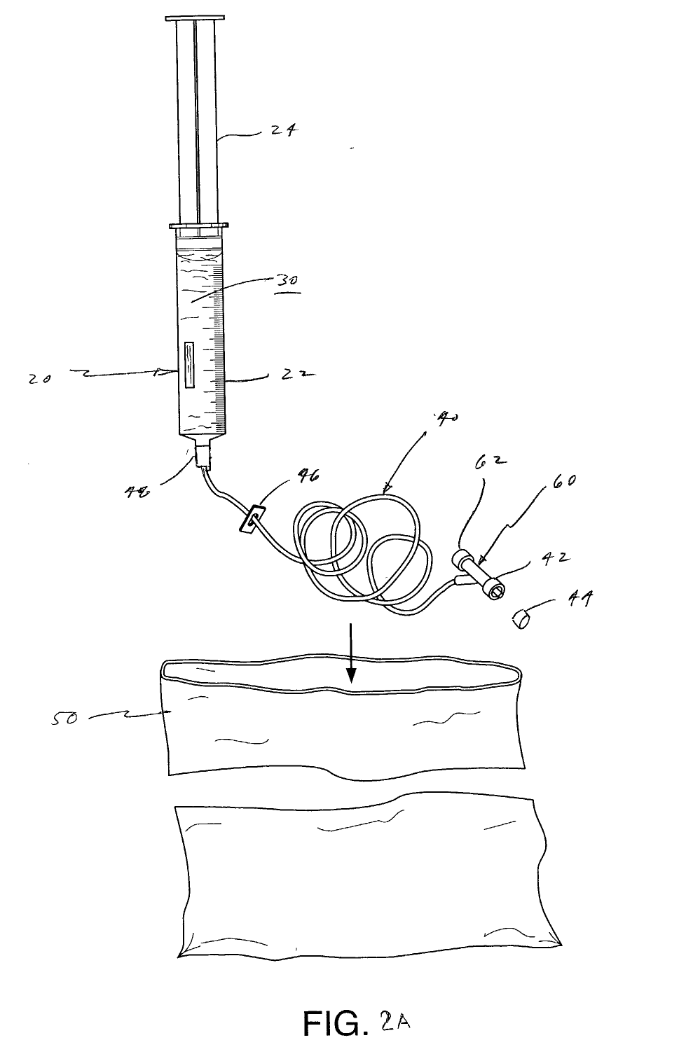

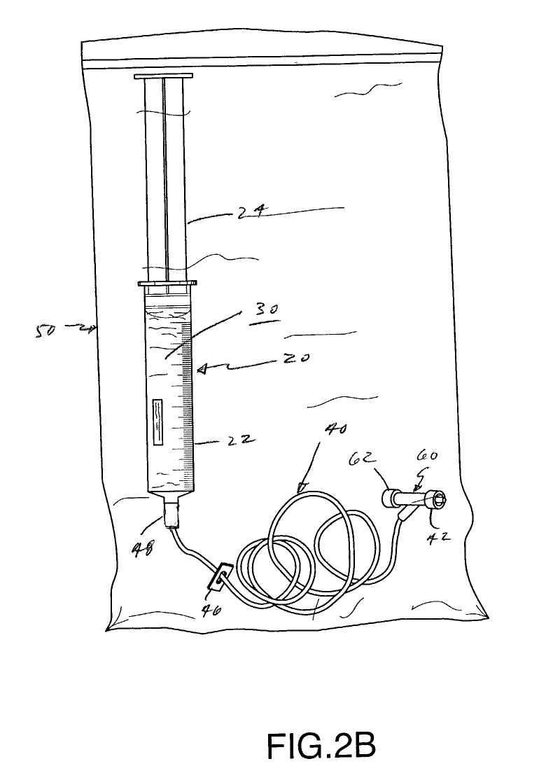

FIGS. 1A, 1B and 2A, 2B illustrate alternate embodiments of a pre-filled syringe assembly 10 comprising the present invention. In each embodiment, the assembly 10 includes a syringe 20 filled with a medical liquid 30, and a tubing length 40 interconnected with or interconnectable to the pre-filled syringe 20. The assembly 10 further includes a packaging enclosure 50 for sealably receiving the pre-filled syringe 20 and tubing length 40.

The syringe 20 includes a barrel 22 and plunger 24 slidably disposed therewithin. As shown in FIG. 1, plunger 24 is located in a retracted position and barrel 22 is filled with medical liquid 30 as packaged. Consequently, assembly 10 may be readily employed by medical personnel when unpackaged. Barrel 22 may be provided with volumetric gradation indicators thereupon and/or a contents description provided thereupon (e.g. via printing and/or labeling). Plunger 24 and barrel 22 may be sized to accommodate a relatively large volume of medical liquid 30, e.g. in one arrangement plunger 24 presents a liquid interface of about 1 in.².

In primary applications, medical liquid 30 may comprise a relatively large volume of a vascular catheter flush solution. More particularly, a volume of at least about 40 ml. of flush solution, and most preferably about 60 ml. of flush solution is provided for use in successive flushing procedures over the course of extended patient care. By way of primary example, the flush solution may be a saline solution or heparin-containing solution.

The tubing length 40 is preferably of a “microbore” type, e.g. having an inside diameter of between about 0.75 mm and 1.50 mm. Further, for common arrangements the tubing length may be between about 30 in. and 75 in., and most typically about 60 inches. The tubing length 40 may be preferably provided with an outlet connector 42 at a distal end thereof, wherein the outlet connector 42 is adapted for ready interconnection with a vascular catheter access port. For example, outlet connector 42 may be a male luer connector sized for screw-on/off interconnection with a complimentary female luer connector defining a vascular catheter access port. To facilitate the visual inspection of fluids therein, outlet connector 42 and/or tubing length 40 may be at least partially transparent. To maintain the sterility of outlet connector 42, a protective cap 44 may also be provided. Further, a line clip 46 (e.g. having a key-hole slot) may be included about tubing length 40 for selective occlusive positioning during use.

As shown, tubing length 40 may be interconnected to barrel 22 prior to packaging. For example, the proximal end of tubing length 40 may be provided with a female luer connector 48 that is rotatably secured to a male luer fitting of syringe 20. Alternatively, the proximal end of tubing length 40 may be fixedly interconnected to syringe 20 prior to packaging.

In one arrangement, the tubing length 40 may be both interconnected to syringe 20 and primed prior to packaging. To prime the tubing length 40, the syringe 20 may be initially overfilled with a volume of medical liquid 30. Then, with cap 44 removed, plunger 24 may be advanced so that a small portion of the medical liquid 30 is dispensed from syringe 20 to prime tubing line 40. Thereafter, cap 44 may be placed on connector end 42 prior to packaging of assembly 10 in enclosure 50.

In another arrangement, the tubing length 40 may be provided with a proximal female luer connector 48 and protective end cap thereupon (not shown), wherein the tubing length 40 is packaged with the pre-filled syringe 30 in a disconnected state. The syringe 30 may be slightly overfilled with medical liquid 30, wherein tubing length 40 may be interconnected to and primed from syringe 20 after unpackaging. More particularly, after unpackaging the protective caps on each end of the tubing length 40 may be removed and the proximal female luer connector 48 connected to the complimentary outlet end of syringe 20. Then, plunger 24 may be advanced so that a small portion of medical liquid 30 is dispensed from syringe 20 to prime the tubing line 40.

In the embodiment of FIGS. 2A, 2B a Y-site member 60 is interconnected (e.g. fixedly bonded) to the distal end of tubing length 40. The Y-site member 60 includes an outlet connector 42 and an inlet connector 62. More particularly, inlet connector 62 may be a female luer connector employable for ready interconnection with a liquid medication source. For example, inlet connector 62 may be provided for ready interconnection with an outlet connector(e.g. a male luer connector) of a tubing line that is interconnected to a multi-dose reservoir of liquid medication (e.g. either directly for use with an infusion pump or via a gravity-fed drip chamber interposed therebetween), or to a multi-dose syringe of liquid medication that is located in an auto-infuser device. Alternatively, the inlet connector 62 may be readily interconnected directly with a syringe containing liquid medication.

As may be appreciated, pre-filled syringe assembly 10 may be sterilized prior to transport, storage and use. In a primary approach, such sterilization may be completed by exposing syringe 20 to gamma radiation after filling with the medical liquid 20. Further, gamma radiation exposure may be utilized for sterilization of tubing length 40. Where tubing length 40 is connected to syringe 20 prior to packaging, the pre-filled syringe 20 and tubing length 40 may be exposed to gamma radiation simultaneously (e.g. after priming of the tubing length 40 as noted above). As will be appreciated, sterilization via gamma radiation exposure may be completed prior to, or after the sealed disposition of pre-filled syringe 20 and tubing length 40 within the packaging enclosure 50, as shown in FIG. 2.

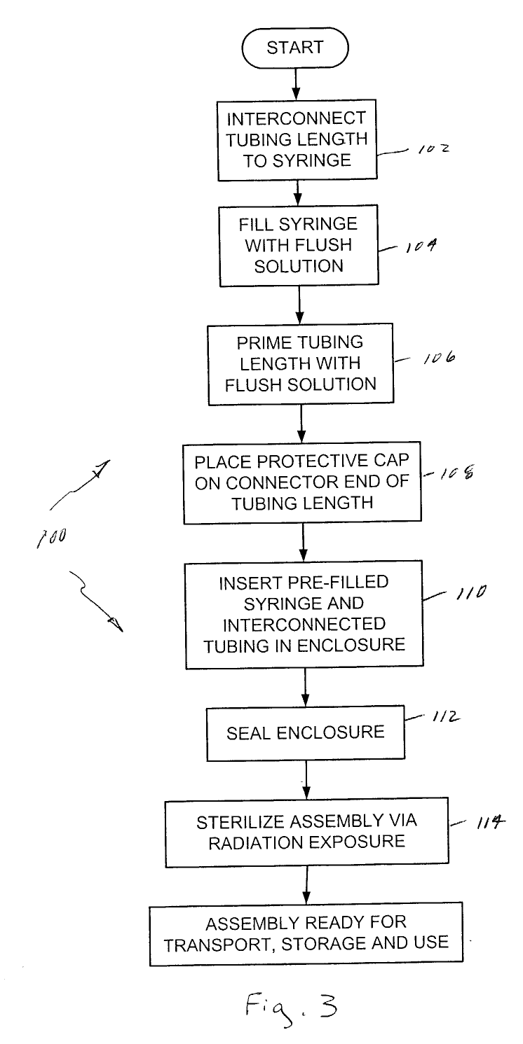

By way of summary, FIG. 3 illustrates steps of an exemplary method 100 for providing the syringe assembly 10 described above. The method 100 optionally provides for the interconnection of a tubing length 40 to syringe 20 (step 102), e.g. via female luer connector 48. The method 100 further includes the step of filling the syringe 20 with flush solution 30 (step 104). In conjunction with such filling, the method may optionally include priming of the tubing length 40 with the flush solution 30 (step 106). As previously indicated, such priming may be achieved by simply advancing the plunger 24 of syringe 20 so as to dispense a small portion of flush solution 30 through tubing length 40. After priming, the method may further provide for the optional placement of a protective cap 44 on an outlet connector 42 of the tubing length 40 (step 108). The pre-filled syringe with interconnected tubing length 40 may then be inserted into the enclosure packaging 50 (step 110). Thereafter, the enclosure packaging 50 may be sealed (e.g. via heat sealing). Then, the assembly 10 may be sterilized via exposure of the packaged assembly to gamma radiation (step 112). After sterilization, the packaged assembly 10 is ready for transport, storage and use.

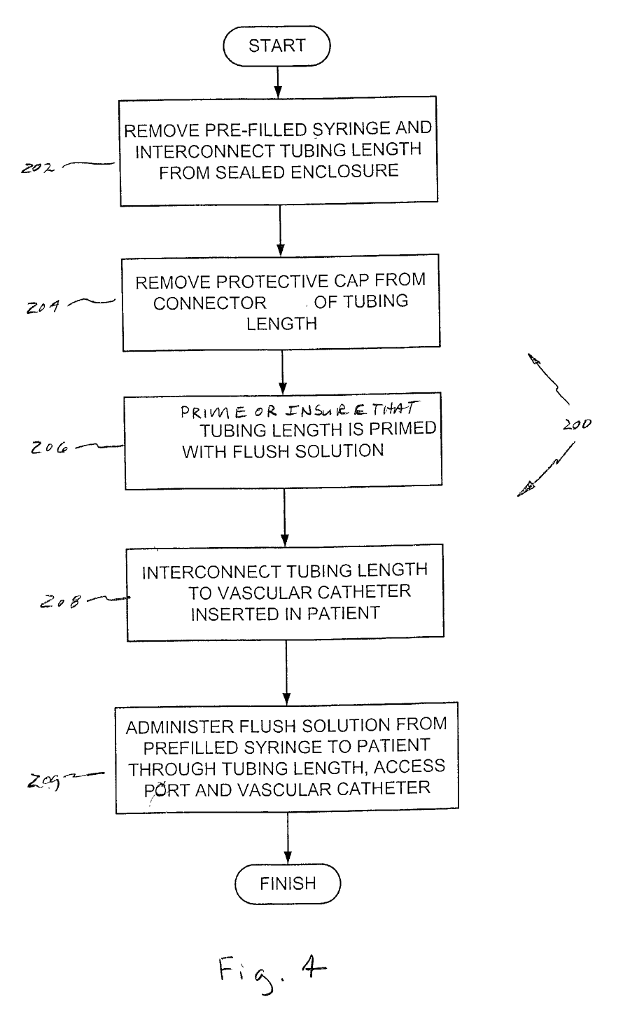

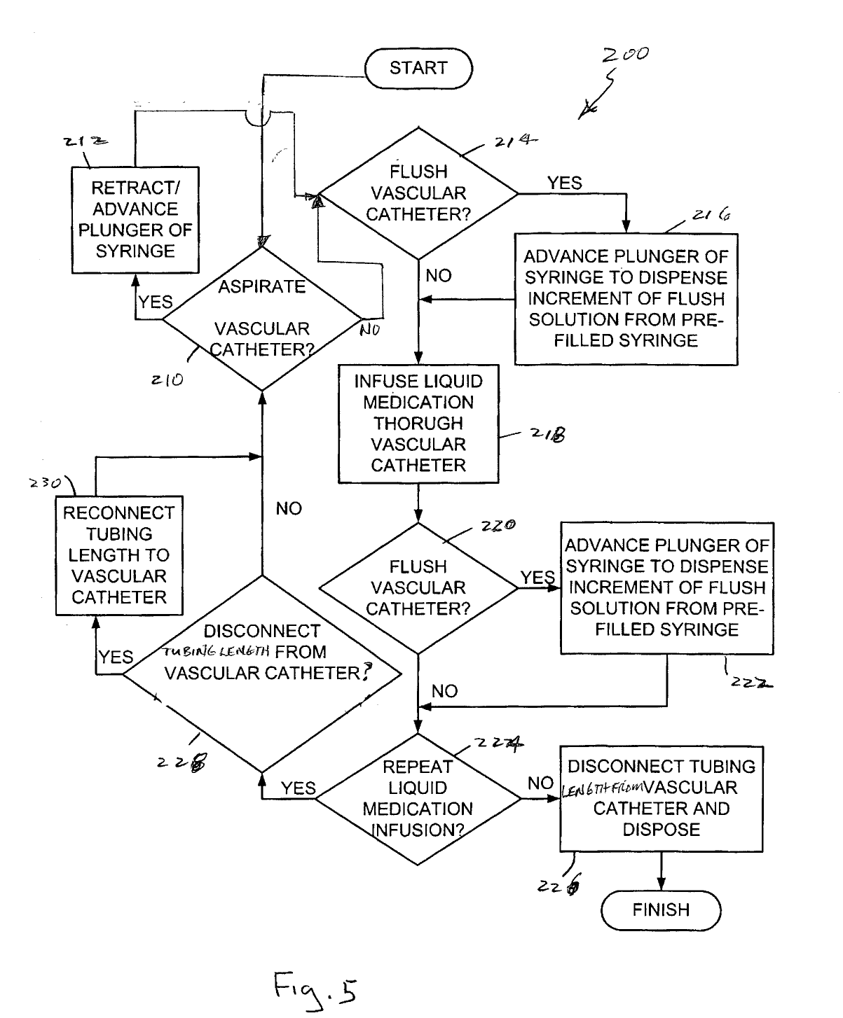

Assembly 10 yields a number of advantages in use. To facilitate an understanding of such advantages reference is now made to FIGS. 4 and 5 which illustrate steps of one method embodiment in which assembly 10 is utilized by medical personnel at a patient care site (e.g. after transport and storage of assembly 10).

With particular reference to FIG. 4, use of assembly 10 may be initiated by removing the pre-filled syringe 20 and an interconnected tubing length 40 from the sealed enclosure 50 (step 202). In embodiments where pre-filled syringe 20 and tubing length 40 are packaged in a disconnected state, the two may be readily interconnected upon removal from enclosure 50 (e.g. via use of a female inerconnector 48 noted above). Next, protective cap 44 may be removed from the outlet connector 42 of tubing length 40 (step 204). To prime or otherwise ensure that the tubing length 40 is properly primed with the flush solution 30, the plunger 24 of pre-filled syringe 20 may be advanced until a small amount of the flush solution 30 is dispensed from the outlet connector 42 (step 206). Additionally, when a Y-site member 60 is provided at the distal end of tubing length 40, the inlet connector 62 thereof may be interconnected to a liquid medication source as noted above. In particular, where such interconnection is made to an outlet connector of a tubing line, such tubing line may be primed from the liquid medication source after interconnection.

At this point, assembly 10 is ready for interconnection to a vascular catheter, e.g. wherein the catheter is already inserted in a patient (step 208). Such interconnection may be made by attachment of the outlet connector 42 to an access port to the vascular catheter (e.g. via a screw-on connection between complementary luer connector ends).

The illustrated method further comprises the step of administering the flush solution 30 from pre-filled syringe 20 to the patient through the tubing length 40 and interconnected vascular catheter (step 209). Of note, the administration of flush solution 30 may entail a number of optional steps carried out in connection with multiple liquid medication infusions and corresponding flush solution administrations over an extended period of patient care. Such options will be described with reference to FIG. 5.

As shown, upon interconnection of assembly 10 to a vascular catheter, medical personnel may want to aspirate the vascular catheter (step 210), e.g. to insure that the vascular catheter is properly inserted into the patient. To do so, plunger 22 of pre-filled syringe 20 may be slightly retracted/advanced to draw/infuse a small portion of blood through the vascular catheter (step 212). Then, medical personnel may desire to flush the vascular catheter with an increment of flush solution 30 from syringe 20 (step 214). To do so, the plunger 24 of syringe 20 may be advanced to dispense the desired increment of flush solution 30 (step 216). Next, medical personnel may infuse a liquid medication through the vascular catheter (step 218). By way of example, the liquid medication may be passed into an inlet connector 62 of a Y-site member 60 provided on tubing length 40. After infusion of the liquid medication through the vascular catheter, medical personnel may selectively flush the vascular catheter (step 220), e.g. to complete infusion of the desired infusion dosage and otherwise reduce subsequent potential blood clotting in the vascular catheter. To do so, plunger 22 of pre-filled syringe 20 may be further advanced to dispense an increment of the flush solution 30 from the syringe 20 (step 222). As may be appreciated, the flushing of vascular catheter as per step 222 may be particularly desirable in situations where the infusion of liquid medication through the vascular catheter is to be repeated (step 224).

When no additional liquid medication infusion is desired, tubing length 40 may be disconnected from the access port to the vascular catheter and assembly 10 properly disposed (step 226). Where further infusion of a liquid medication is desired, medical personnel may optionally determine whether tubing length 40 is to be disconnected from the vascular catheter (step 228), e.g. to facilitate patient movement in the interim time period before the next infusion of liquid medication. When the tubing length 40 is disconnected, the tubing length 40 will need to be selectively reconnected to the vascular catheter prior to the next liquid medication infusion (step 230). However, it should be appreciated that, when a Y-site member 60 is utilized, the interconnections between the Y-site member 60 and syringe 20 and between the Y-site member 60 and a liquid medication source may be maintained, i.e. while outlet connector 42 is disconnected from the vascular catheter access port.

Prior to the next medical liquid infusion, medical personnel may desire to aspirate the vascular catheter (step 210), e.g. to insure that the vascular catheter is properly located and/or to insure that a blood clot has not formed within the vascular catheter. To do so, plunger 22 of syringe 20 may be slightly retracted/advanced so as to draw/return a small portion of blood through the vascular catheter (step 212). It should be noted that such aspiration is particularly facilitated by an administration apparatus described hereinbelow.

Prior to the further infusion of liquid medication through the vascular catheter, medical personnel may again desire to flush the vascular catheter thereof (step 214). For such purposes, plunger 22 of syringe 20 may again be advanced to dispense another increment of flush solution 30 from the syringe 20 (step 216). Thereafter, medical liquid infusion and further flushing may continue in a repeated fashion as indicated by FIG. 5.

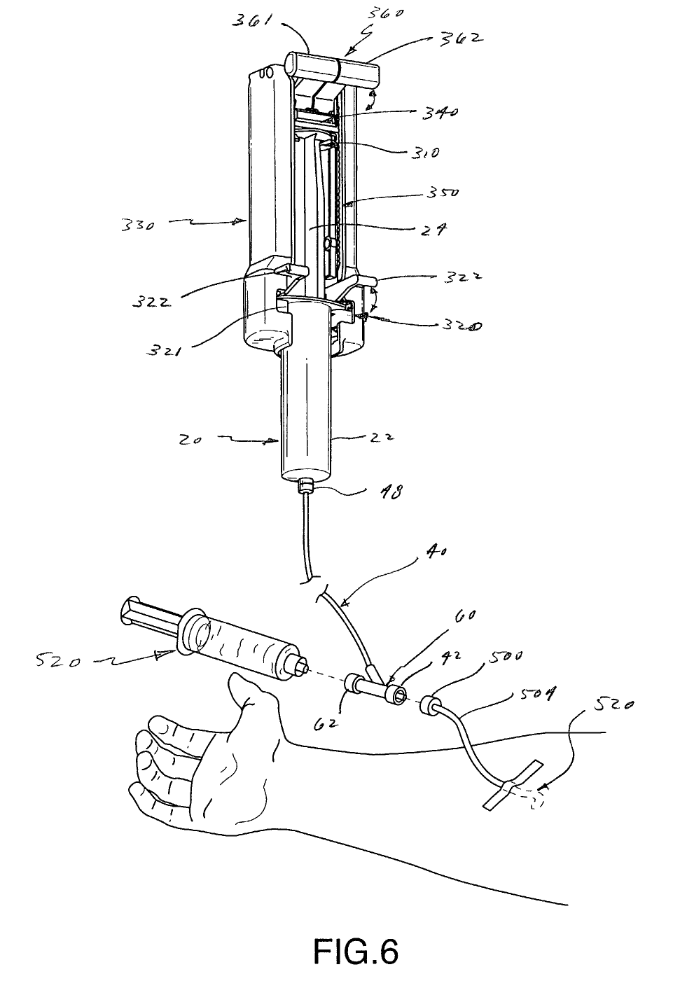

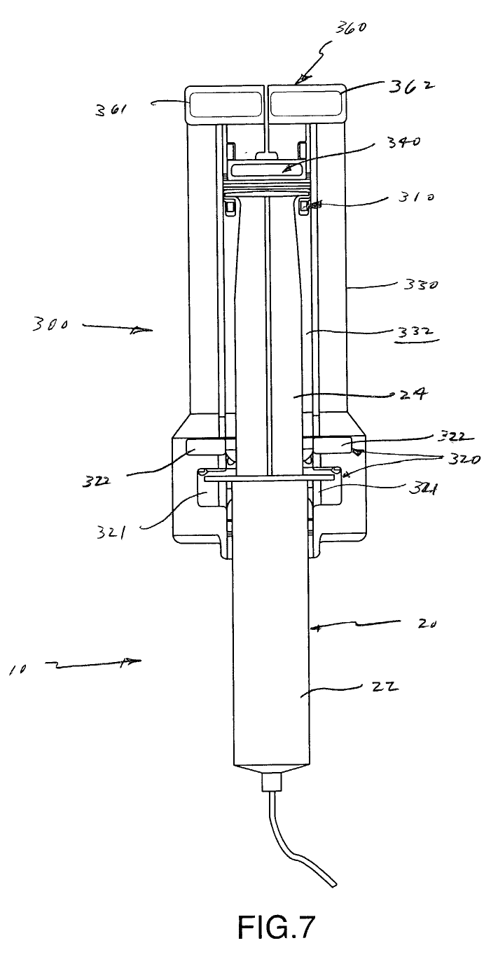

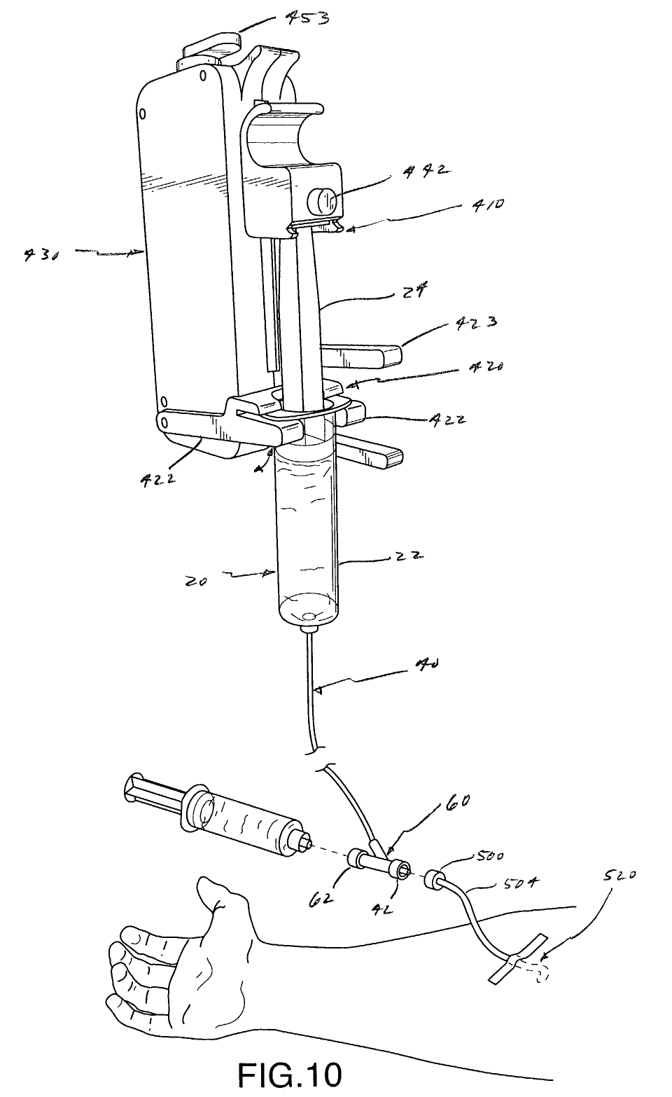

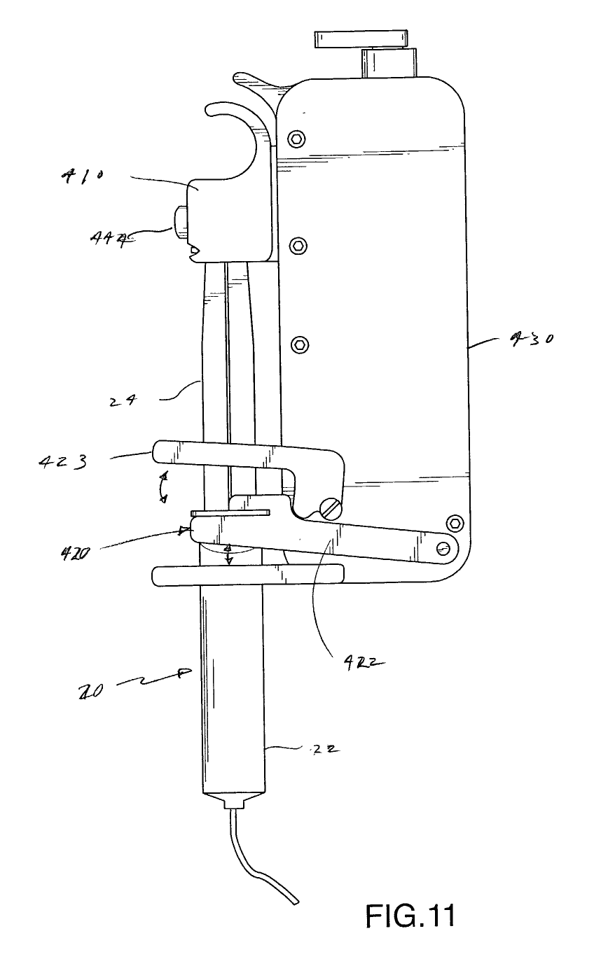

Reference is now made to FIGS. 6-9 and FIGS. 10-13 which illustrate alternate embodiments 300 and 400 of an apparatus for use in the administration of flush solution 30 of syringe assembly 10. As will be appreciated, apparatus embodiments 300 and 400 may be advantageously employed to facilitate and otherwise simplify the execution of the various steps described in relation to FIG. 5 above. In FIGS. 6 and 10, syringe assembly 10 is shown loaded in the administration apparatus 300 and 400, respectively, with outlet connector 42 positioned for ready, rotatable connection to a female luer access port 500 that is fluidly connected by a tubing line 504 to a vascular catheter 510 inserted into the arm of a patient. Further, an inlet connector 62 of a Y-site member 60 is positioned for interconnection to a liquid medication source such as the illustrated syringe 520. Of course, other liquid medication source arrangements may also be employed. In particular, multi-dose liquid medication sources may be interconnected via a tubing line having an outlet connector (e.g. a male inerconnector).

As shown in FIGS. 6-9, administrative apparatus 300 includes first and second holder members 310, 320 for holding the plunger 24 and barrel 22 of syringe 20, respectively. The first and second holder members 310, 320 are disposed relative to a housing 330 in a manner that facilitates controlled relative movement therebetween, thereby providing for the metered administration of flush solution 30 from syringe assembly 10.

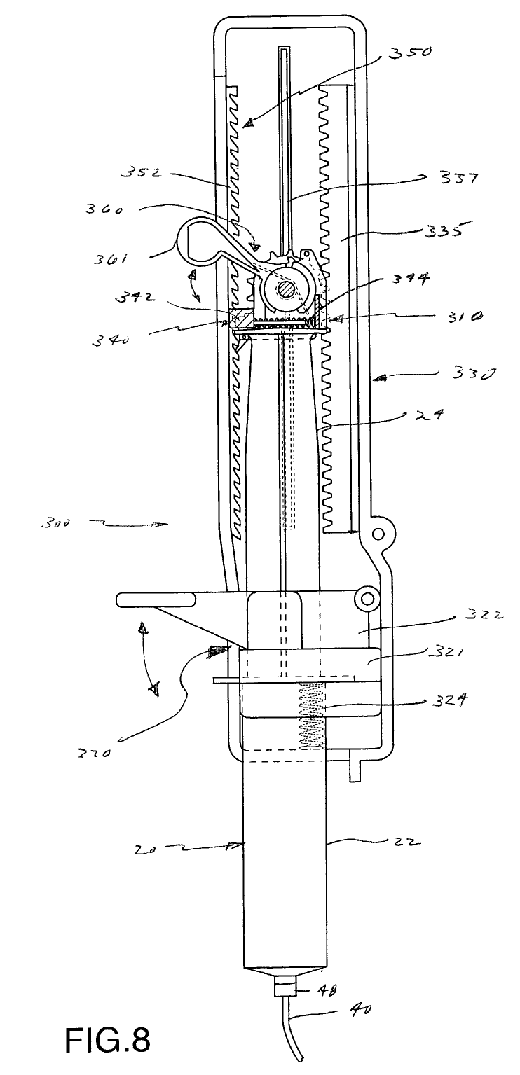

More particularly, the first holder member 310 supports a catch assembly 340 that is adapted to successively engage a plurality of stops 350 provided with housing 330 along a travel path of the first holder member 310. As best shown by FIG. 8, the plurality of stops 350 may be defined by downwardly angled teeth of a stop member 352 disposed on one side of a housing slot 332 along which first holder member 310 travels during use. Another stop member of common configuration (not shown) may be provided on the other side of housing slot 332.

The catch assembly 340 includes a catch member 342 and at least one spring 344 that spring-loads the catch member 342 towards the noted plurality of stops 350. As such, when catch member 342 is in spring-loaded engagement with one of the plurality of stops 350, movement of the first holder member 310 away from the second holder member 320 is restricted. Preferably, spring(s) 344 provide a spring-loading of between about 2 psi and 5 psi, and most preferably about 3 psi.

Administrative apparatus 300 also includes a drive assembly 360 supported by first holder member 310 and having first and second drive arms 361, 362 for driving the first holder member 310 towards the second holder member 320, as will be further discussed. Further, the second holder member 320 is spring-loaded towards the first holder member 310. More particularly, the second holder member 320 includes opposing, U-shaped bottom and top members 321, 322, respectively. The side arms of the bottom member 322 are configured to define side shelves, or seats, which together with the top member 321 combinatively define a slot that is configured to support and retain a complimentary flange located at the top end of the barrel 22 of syringe 20. In this regard, the outside of each side arm of the bottom member 322 may be notched to slidably engage guide tracks (not shown) on the housing 330 and may be spring-loaded towards the top member 321 by one or more coil spring(s) 324 to assume a biased “home” position as shown in FIGS. 6-9. At the home position movement of the top member 322 away from the spring-loading may be restricted by the housing 330. Preferably, coil spring(s) 324 apply a spring force of between about 5 psi and 15 psi to the second holder when attached. Further, such spring force is preferably relatively constant across the intended actuation range.

When one of the drive arms 361, 362 of drive assembly 360 is employed to advance the first holder member 310 and plunger 24 towards the second holder member 320 and barrel 22, the second holder member 320 and barrel 22 may move downward and away from the first holder member 310 and plunger 24 against the noted spring-loading. Such movement of the second holder member 320 allows the catch assembly 340 to engage one of the plurality of stops 350. Then, second holder member 320 moves back into its home position in response to coil spring(s) 324 as plunger 24 moves towards barrel 22 to effect the metered administration of flush solution 30 from the outlet of barrel 22.

In this regard, it should be appreciated that the amount of flush solution 30 metered will be a direct relation to the amount of linear travel of the first holder member 310 between the different locations, or stop positions, at which catch assembly 340 restrainedly engages different ones of the plurality of stop members 350. As such, the first and second drive arms 361, 362 may be provided to effect differing amounts of linear travel of the firsts holder 310 when pivoted. In turn, differing increments of flush solution administration may be realized.

Of further note, the top member 322 of the second holder number 320 may be pivotably connected along its back to housing 330 and may include lever arms on each side that extend outward beyond housing 330 for convenient control by a user. Specifically, such lever arms may be manually pivoted towards the bottom member 322 by a user against the noted spring-loading, wherein the coil spring(s) 324 is compressed. As may be appreciated, this feature allows a user to selectively aspirate a vascular catheter 520 that is interconnected to a pre-filled syringe 20. For example, a user may slightly pivot the top member 322 against the spring-loading, wherein the second holder member 320 moves the barrel 24 of syringe 20 away from the plunger 22 held by the first holder member 310. Such movement may be controlled with a high degree sensitivity to draw blood from a patient through a vascular catheter 520 connected to pre-filled syringe 20. In turn, upon release of the lever arms, the spring-loaded second holder member 320 will assume its home position as the blood is returned to the patient.

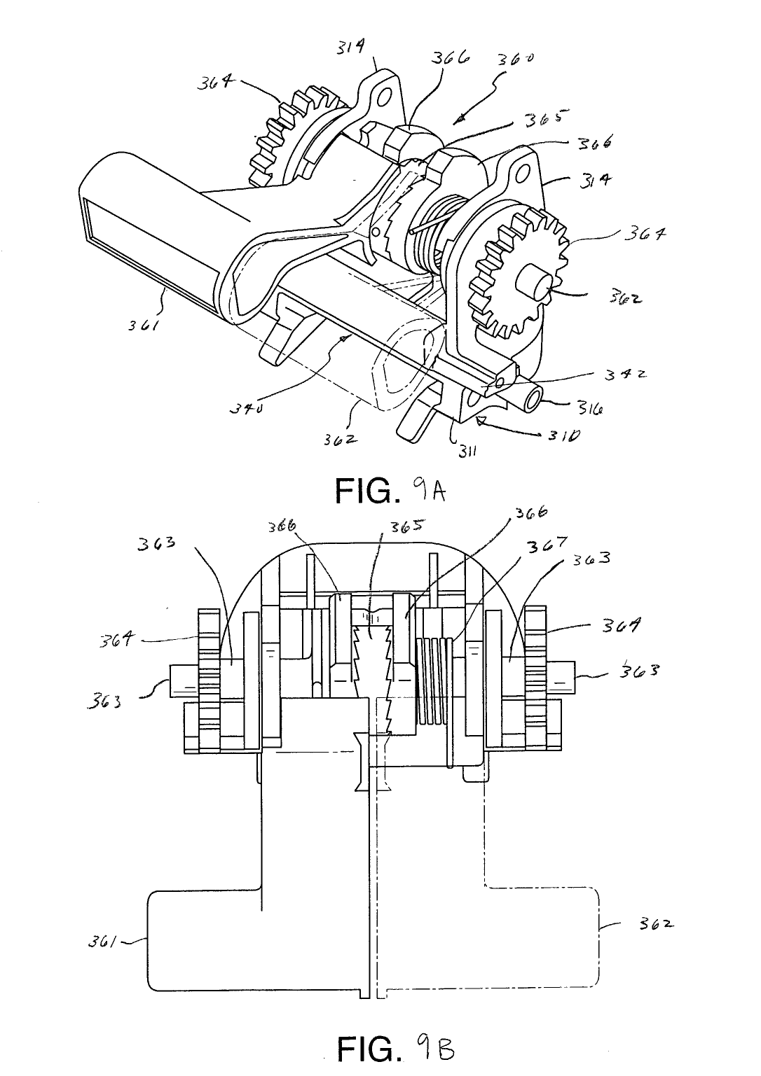

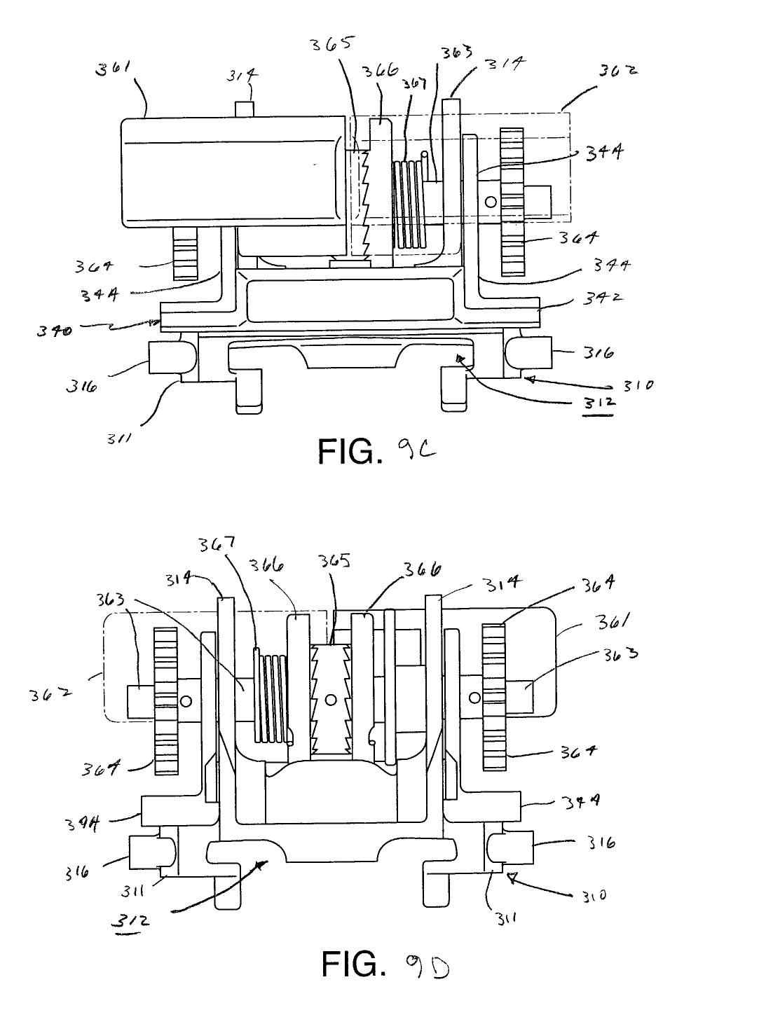

Reference is now made to FIGS. 9A, 9B, 9C and 9D which illustrate in greater detail the first holder member 310, catch assembly 340 and drive assembly 360. For purposes of illustration, the second drive arm 362 of drive assembly 360 is shown in phantom lines.

First holder member 310 includes a bottom portion 311 having a slot 312 configured to receive a complimentary at the top end of plunger 24 of pre-filled syringe 20. Additionally, the first holder member 310 includes an upstanding yoke portion 314 to which the catch assembly 340 and drive assembly 360 are supportably mounted. The first holder member 310 also includes arms 326 extending on each side and configured for sliding receipt within slots 337 provided on each side of the housing 330 (see FIG. 8).

Drive assembly 360 includes a shaft member 363 that is rotatably carried by the yoke portion 314 of the first holder member 310. Each end of the shaft member 363 is configured for sliding receipt positioning within the slots 337 of the housing 330. Further, toothed drive wheels 364 are interconnected near each end of shaft member 363 and disposed to rotatably engage corresponding drive tracks 335 disposed within housing 330 along the travel path of the first holder member 320.

Drive assembly 360 further includes a central ratchet member 365 interconnected to shaft member 363 for co-rotation therewith. Additionally, outer ratchet members 366 are rotatably carried by shaft member 363 on opposing sides of central ratchet members 365. Each of the outer ratchet members 366 are interconnected to a corresponding one of the drive arms 361, 362, respectively, for co-rotation therewith. The outer ratchet members 366 are also disposed in spring-loaded engagement with central ratchet member 365 by corresponding coil springs 367 mounted on shaft member 363. For example, one end of each coil springs 367 may be interconnected to a corresponding, adjacent outer ratchet member 366 while the other end of the coil spring 367 is retainably positioned within a slot of the corresponding drive arm 361 or 362.

Catch assembly 340 includes support arms 342 which are rotatably interconnected to and supported by shaft member 363. The above-noted spring member(s) 344 is disposed between catch member 342 and the yoke portion 314 of the second holder member 320.

The pivot range of drive arms 361, 362 is restricted to define a corresponding maximum, or full stroke length for each, wherein such full stroke lengths may be established to be different. In turn, differing predetermined increments or amounts of flush solution 30 may be administered from syringe 20 when drive arms 361, 362 are separately pivoted. For example, in one arrangement the full stroke lengths may be set to provide for selective administration increments of 2.5 ml. and 5 ml.

A brief summary of the use of administration apparatus with 300 syringe assembly 10 will now be provided. Initially, a user may remove the pre-filled syringe 20 and tubing length 40 from a sealed enclosure 50, e.g. at a patient care site. Then, the user may locate the first holder member 310 at an appropriate stop position along the plurality of stops 350, e.g. via manual depression of the catch member 342 and movement thereof with the first holder member 310 in housing slot 332. Subsequently, the user may load the pre-filled syringe 20 into the administration apparatus 300. To do so, the flange of barrel 24 is properly oriented and slid into the slot of the second holder member 320 and the flange of the plunger 22 is properly oriented and slid into the slot of the top holder member 310.

At this point, it may be pointed out that the administration apparatus 300 may be provided so that when the first holder member 310 is located at a predetermined stop position, e.g. a stop position furthest from the second holder member 320, the spacing between the first holder member 310 and second member 320 corresponds with the spacing between the flanges of barrel 24 and plunger 22 of pre-filled syringe 20. Such complimentary design further facilitates use of the syringe assembly 10 and administration apparatus 300.

Prior to or after loading of the pre-filled syringe 20 into the administration apparatus 300, the user may insure the interconnection of tubing length 40 with pre-filled syringe 20 and prime the same. After priming the tubing length 40 may be fluidly interconnected to a vascular catheter 520 inserted into a patient, e.g. via an outlet connector 42 and vascular catheter access port 500.

To administer the flush solution 30, a user simply pivots drive arm 361 or 362, depending on the desired increment. To aspirate the vascular catheter 520, a user simply pivots one of the lever arms of the second holder member 320. Such steps may be repeated as desired to facilitate the various method steps described hereinabove, e.g. in connection with FIG. 5.

Referring now to FIGS. 10-13, administration apparatus 400 includes first and second holder members 410, 420 for holding the barrel 22 and plunger 24 of syringe 20, respectively, relative to a dispenser housing 430. The first and second holder members 410, 420 are disposed relative to a housing 430 in a manner that again facilitates controlled relative movement therebetween, thereby providing for the metered administration of flush solution 30 from syringe assembly 10.

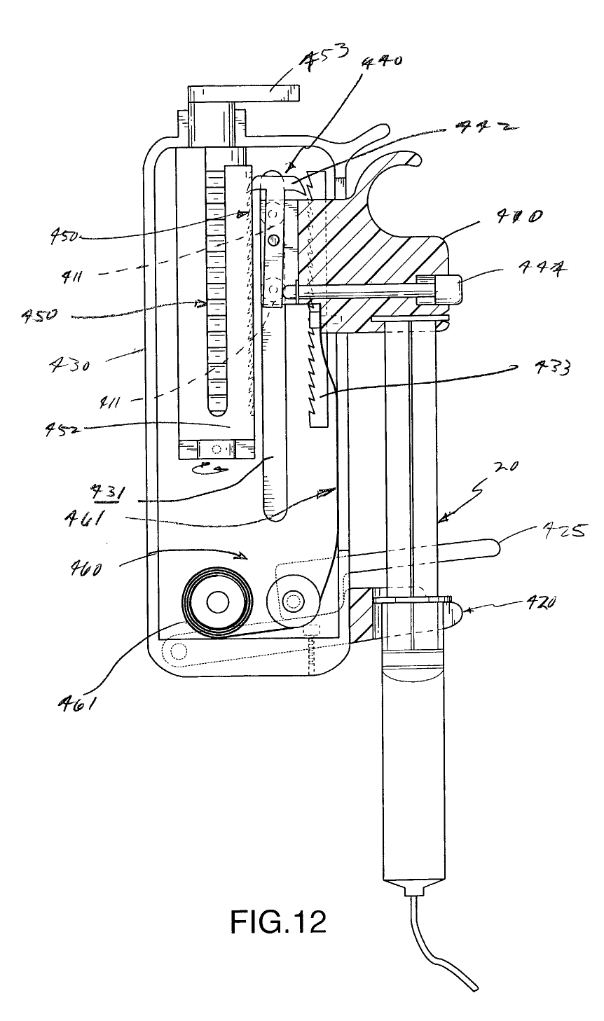

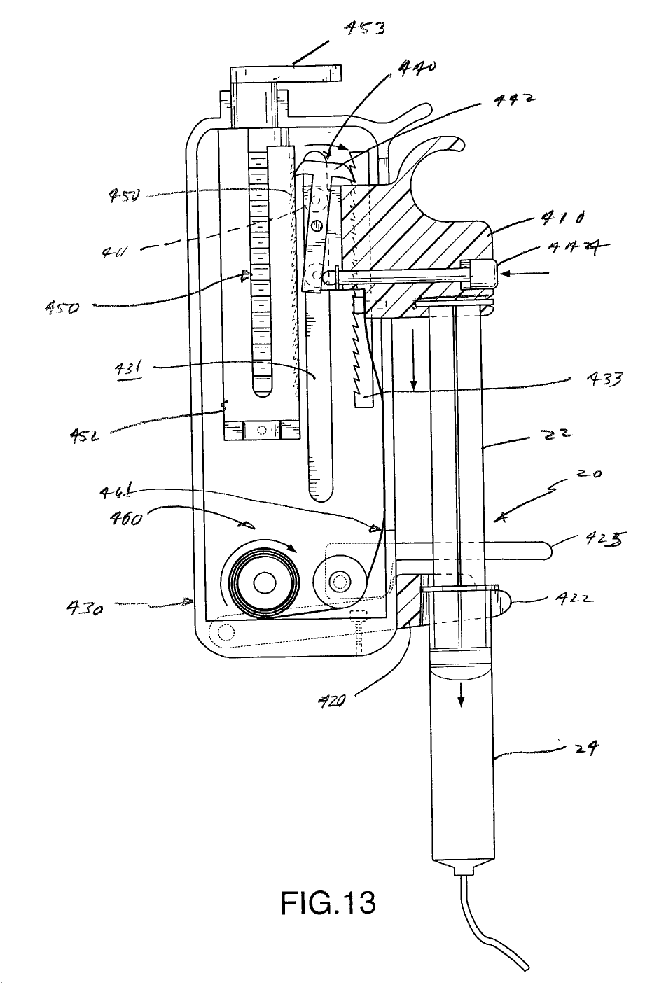

In this regard, the first holder member 410 supports a catch assembly 440 that is adapted to successively engage a plurality of stops 450 provided by housing 430 along a travel path of the first holder member 410. As best shown by FIGS. 12 and 13, the plurality of stops 450 may be defined by upwardly angled teeth of a stop member 452 disposed parallel to a housing slot 432 along which the first holder member 410 travels during use. The stop member 452 may be selectively moved within housing 430 to present different sets or columns of stops 450, each having different stop spacings. For example, the stop member 452 may be journaled to housing 430 so that a selection lever 453 may be rotated to rotate the desired column of stops 450 into position for engagement with catch assembly 440. As will be appreciated, the selected column of stop members 450 will establish the increment or amount of flush solution 30 administered when the first holder member 410 is between successive stop positions.

The catch assembly 440 includes a catch member 442 in the form of a pawl that is pivotably interconnected to the first holder member 410 and spring-loaded towards the noted plurality of stops 450, e.g. wherein the spring-loading is preferably between about 2 psi and 5 psi, and most preferably about 3 psi. As such, when catch member 442 is in spring-loaded engagement with one of the plurality of stops 450, movement of the first holder member 410 towards the second holder member 420 is restricted. As shown, the catch assembly 440 also includes a selectively depressible actuator 444 that functions to pivot the catch member 442 against the spring-loading and out of engagement with the plurality of stops 450, thereby allowing for movement of the first holder member 410 relative to the second holder member 420. To help maintain the desired orientation of the first holder member 410, side arms 411 may be provided on either side to follow complimentary recess tracks 431 provided by housing 430. As also shown, a toothed track 433 is disposed in opposing relation to the plurality of stops 450 to engage catch member 442 and thereby facilitate controlled movement of the first holder member 410.

In this regard, the administration apparatus 400 includes a drive assembly 460 for driving the first holder member 410 towards the second holder member 420. The drive assembly 460 includes a self-winding spring 461 (e.g. a negator-type spring). More particularly, the spring 461 is anchored to housing 430 at its wound end and interconnected to the first holder member 410 at its free end to pull the first holder member 410 towards the second holder member 420 upon a single activation, i.e. a single unwinding which occurs upon loading of a pre-filled syringe 20. Preferably, a spring-loading of between 5 and 15 psi is provided by spring 461.

As may be appreciated, after loading of a pre-filled syringe 20, actuator 444 may be pushed to pivot catch member 442 out of engagement with one of the plurality of stops 450 and into engagement with toothed track 433 as the first holder member 410 is pulled toward the second holder member 420. Upon release of the actuator 444, the spring-loaded catch member 442 will once again pivot to engage another one of the plurality of stops 450. Such sequence effects the administration of one increment of flush solution 30.

The second holder member 420 of administration apparatus 400 is preferably spring-loaded towards the first holder member 410. More particularly, the second holder member 420 is pivotably connected by lever arms 422 to housing 430 and spring-loaded by one or more spring(s) (not shown) located along its pivot axis to assume a “home” position as shown in FIGS. 10-13. Lever arms 422 extend beyond housing 430 and are accessible for manual operation. Further, another arm 425 is pivotably mounted to housing 430 so that it engages, and thereby pivots lever arms 422 when manually pivoted. Such feature may be utilized for vascular catheter 520 aspiration, as previously discussed.

The embodiments described above are for exemplary purposes only and are not intended to limit the scope of the present invention. Various adaptations, modifications and extensions of the embodiment will be apparent to those skilled in the art and are intended to be within the scope of the invention as defined by the claims which follow.

What is claimed:

1. An apparatus for administering a medical liquid from a syringe, comprising:
a first holder member for restrainably engaging a plunger of a syringe;
a second holder member for restrainably engaging a barrel of a syringe, wherein said second holder member is spring-loaded towards said first holder member; and,
a drive means for mechanically driving said first holder member towards said second holder member along a travel path.

2. An apparatus as recited in claim 1, wherein said spring-loading of said second holder member is between about 5 psi and 15 psi.

3. An apparatus as recited in claim 1, further comprising:
at least one spring member for providing said spring-loading of said second holder member towards said first holder member;
a housing to which said second holder member is interconnected for pivotable movement against said spring member; and,
at least one lever arm for manually pivoting said second holder member against said spring-loading of said second holder member.

4. An apparatus as recited in claim 1, further comprising:
stop means for restricting movement of said first holder member at a plurality of spaced stop positions along said travel path.

5. An apparatus as recited in claim 4, wherein stop means includes:
a plurality of stops corresponding with said plurality of stop positions; and,
at least one catch member for restrainably and successively engaging said plurality of stops.

6. An apparatus as recited in claim 5, wherein said plurality of stops are located along said travel path, and wherein said at least one catch member is interconnected to said first holder member and spring-loaded towards said plurality of stops.

7. An apparatus as recited in claim 6, wherein said spring-loading of said catch member is between about 2 psi and 5 psi, and wherein said catch member is manually disengageable relative to said plurality of stops.

8. An apparatus as recited in claim 6, wherein said catch member is a pawl pivotably interconnected to said first holder member, and wherein said stop means further includes:
a manually-actuated push member for pivoting said pawl away from said plurality of stops against said spring-loading, thereof.

9. An apparatus as recited in claim 4, wherein said drive means includes:
a drive spring activatable to an apply spring force to said first holder member to effect movement thereof across said plurality of stop positions upon a single activation of said drive spring.

10. An apparatus as recited in claim 9, wherein said drive spring is disposed to be in tension upon said activation.

11. An apparatus as recited in claim 10, wherein said drive spring is a negator type spring.

12. An apparatus as recited in claim 9, wherein said drive spring is disposed to be in compression upon said activation.

13. An apparatus as recited in claim 4, wherein said drive means includes:
a first drive arm pivotably carried by said first holder member to effect movement of the first holder member between successive ones of said plurality of stop positions.

14. An apparatus as recited in claim 13, wherein said drive means further includes:
a drive shaft rotatably supported by said first holder member; and,
a ratchet means, supported by said drive shaft and interconnected to said first drive arm, for translating pivotal movement of said first drive arm between first and second positions to rotational movement of said drive shaft.

15. An apparatus as recited in claim 14, wherein said ratchet means is further provided to automatically return said first lever arm from said second position to said first position without rotating said drive shaft.

16. An apparatus as recited in claim 13, wherein said drive means further includes:
a drive shaft supported by said first holder members; and,
at least one drive wheel connected to said drive shaft.

17. An apparatus as recited in claim 16, further comprising:
a housing; and,
a drive track interconnected to said housing, for engaging drive wheel along said travel path.

18. An apparatus as recited in claim 5, wherein one of said drive means and said stop means is controllable to establish the spacing between said plurality of stop positions.

19. An apparatus as recited in claim 18, wherein said drive means includes:
a first drive arm pivotably carried by said first holder member to effect movement of the first holder member across a first predetermined number of said plurality of stop positions upon a full pivot stroke thereof; and,
a second lever arm pivotably carried by said second holder member to effect movement of the second holder member across a second predetermined number of said plurality of stop positions upon a full pivot stroke thereof, wherein said first and second predetermined numbers are different.

20. An apparatus as recited in claim 19, wherein said drive means further includes:
a drive shaft rotatably supported by said first holder member; and,
a ratchet means, supported by said drive shaft and separately interconnected to said first drive arm and second drive arm, for translating pivotal movement of said first drive arm and pivot movement of said second drive arm to rotational movement of the drive shaft.

21. An apparatus as recited in claim 18, wherein said stop means includes:
a plurality of sets of stops, wherein any one of said plurality sets is selectively positionable to engage said at least one catch member, and wherein the spacing between adjacent stops is different for each if said plurality of sets.

22. A method for use in administering a medical liquid from a syringe, comprising:
positioning a plunger and barrel of a syringe in a first holder member and a second holder member, respectively:
first driving said first holder member towards said second holder member along a travel path; and,
second driving said second holder member towards said first holder member in response to said first driving step.

23. A method as recited in claim 22, wherein said first driving step includes:
ratcheting said first holder member relative to a drive track positioned along a travel path.

24. A method as recited in claim 23, wherein said ratcheting steps includes:
pivoting first drive arm from a first position to a second position to ratchet said first holder member a first distance.

25. A method as recite in claim 23, wherein said ratchet step includes:
pivoting one of a first drive arm and second drive arm to ratchet said first holder one of a first distance and second distance, respectively, wherein said first and second distances are different.

26. A method as recited in claim 22, wherein said first driving step includes:
applying a spring force to said second holder member.

27. A method as recited in claim 26, further comprising:
actuating said spring force; and,
repeating said first driving and second drive steps a plurality of times during said actuating step.

28. A method as recited in claim 22, further comprising:
restricting movement of said first holder member toward said second holder member at a plurality of spaced stop positions along said travel path.

29. A method as recited in claim 25, wherein said restricting step includes:
engaging a catch member with one of a plurality of stops corresponding with said plurality of stop positions;
disengaging said catch member from said one of said plurality of stops; and,
repeating said engaging and disengaging a plurality of times, wherein each time the catch member engages and disengages a different one of said plurality of stops.

30. A method as recited in claim 24, wherein said disengaging step includes:
manually applying a force of between about 2 psi and 5 psi to said catch member.

31. A method as recited in claim 22, wherein said second driving step includes:
applying a spring force to said second holder member.

32. A method as recited in claim 31, wherein said spring force is between about 5 psi and 15 psi.

33. A method as recited in claim 22, further comprising:
manually moving said second holder member away from said first holder member; and,
third driving said second holder member towards said first holder member and response to said manually applying step.

34. A method as recited in claim 33, wherein said third driving steps includes:
restricting movement of said first holder member toward said second holder member at a plurality of spaced stop positions along said travel path.

35. A method as recited in claim 31, wherein said restricting step includes:
engaging a catch member with one of a plurality of stops corresponding with said plurality of stop positions;
disengaging said catch member from said one of said plurality of stops; and,
repeating said engaging and disengaging a plurality of times, wherein each time the catch member engages and disengages a different one of said plurality of stops.

36. A method as recited in claim 33, wherein said manually moving step includes:
pivoting said second holder member away from said first holder member.
